(19) United States
(12) Patent Application Publication
(10) Pub. No.: US 2014/0350378 A1
(43) Pub. Date: Nov. 27, 2014

(54) Method for Classifying Tissue Response to Cancer Treatment Using Photoacoustics Signal Analysis

(71) Applicants: Behnaz Pourebrahimi, Toronto (CA); Michael Christopher Kolios, Toronto (CA); Gregory Jan Czarnota, Toronto (CA)

(72) Inventors: Behnaz Pourebrahimi, Toronto (CA); Michael Christopher Kolios, Toronto (CA); Gregory Jan Czarnota, Toronto (CA)

(21) Appl. No.: 14/169,421

(22) Filed: Jan. 31, 2014

Related U.S. Application Data
(60) Provisional application No. 61/759,525, filed on Feb. 1, 2013.

(51) Int. Cl. A61B 5/00

(52) U.S. Cl. CPC A61B 5/0095
USPC 600/407

(57) ABSTRACT

A method for monitoring tissue response to cancer treatment by analyzing photoacoustic signals is provided. The method is capable of classifying different levels of tumor response for a tumor under treatment. Photoacoustic signals are obtained by scanning a tumor before and after treatment in given time intervals. Classification of the tumor response is achieved based on statistical features extracted from the photoacoustic signals. The similarities and dissimilarities between the statistical features are used to identify and classify the changes in tissue as the tumor responds to the treatment. To visualize the similarities and dissimilarities, a computed similarity metric is mapped to a multidimensional space, such as a two- or three-dimensional space. In this produced tissue response map, each point represents the tumor in a particular condition. Points that are farther apart in the tissue response map indicate more changes in tumor tissue due to changing condition and vise versa.

### CROSS-REFERENCE TO RELATED APPLICATIONS

This application claims the benefit of U.S. Provisional Patent Application Ser. No. 61/759,525 filed on Feb. 1, 2013.

### BACKGROUND OF THE INVENTION

The field of the invention is systems and methods for photoacoustic imaging. More particularly, the invention relates to systems and methods for monitoring cancer treatment using photoacoustic signal analysis.

Cancer patients do not respond identically to certain treatment regimes. As such, a predefined therapy is not often practically effective for all patients. This makes the early detection of patients' refractory to a specific therapy critical, since it could facilitate the switch to an early salvage therapy. Standard anatomical-based imaging can detect macroscopic changes in tumor size as a measure of treatment response. Such changes often take many weeks to months to develop, however, and in some cases tissue diminishment is not present despite a positive treatment response.

Functional imaging methods including magnetic resonance imaging (“MRI”) and positron emission tomography (“PET”) have been demonstrated as being capable of detecting tumor responses early after starting therapy. Such methods, which frequently probe tumor physiology, could be used to navigate changes in treatment non-invasively in order to optimize final prognosis.

Photoacoustic imaging has the advantage of low cost, rapid imaging speed, portability, and high resolution. Moreover, unlike the other modalities being investigated for treatment monitoring, no injections of contrast agents or radiopharmaceuticals are needed because the image contrast changes are caused by changes in the physical properties of dying cells.

It would therefore be desirable to provide a method for monitoring the response of cancerous tissue to treatment using photoacoustic imaging techniques.

### SUMMARY OF THE INVENTION

The present invention overcomes the aforementioned drawbacks by providing a method for monitoring physical changes in a tissue in response to a treatment using a photoacoustic measurement system, such as a photoacoustic spectroscopy or imaging system. Photoacoustic data is acquired from one or more locations within the tissue in both a first condition and a second condition that is different than the first condition. For instance, the first condition can be a time before treatment and the second condition can be a time after treatment. Similarity metrics between the photoacoustic signals acquired in the different conditions are computed. The photoacoustic data acquired in the different conditions are then mapped into a multidimensional space using the computed similarity metrics, thereby producing a treatment response map. The first condition and second condition are classified as corresponding to a particular treatment response by comparing locations of the mapped photoacoustic data in the treatment response map.

The foregoing and other aspects and advantages of the invention will appear from the following description. In the description, reference is made to the accompanying drawings which form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

FIG. 1 is a flowchart setting forth the steps of an example of a method for monitoring and classifying tissue response to a treatment by analyzing photoacoustic signals acquired from the tissue in different conditions;

FIG. 2A is an example plot that depicts the visualization of similarity patterns in data frames acquired for a “pre-treatment” condition and a “post-treatment” condition in a control study where no treatment has been administered;

FIG. 2B is an example plot that depicts the visualization of similarity patterns in data frames acquired for a pre-treatment condition and a post-treatment condition when a treatment has been administered; and

FIG. 3 is a block diagram of an example of a photoacoustic imaging system that includes a photoacoustic device.

### DETAILED DESCRIPTION OF THE INVENTION

Described here are systems and methods for monitoring cancer treatment effects by visualizing a cancerous tumor in different stages of treatment by way of analyzing photoacoustic (“PA”) signals. The method is capable of classifying different levels of tumor response for the tumor under treatment.

In general, the method of the present invention is carried out as follows. PA signals are obtained by scanning a tumor before and after treatment in given time intervals. For instance, the tumor can be scanned at successive times post-treatment to obtain information pertaining to the longer-term tumor response to the treatment. Classification of the tumor response is achieved based on statistical features extracted from the PA signals in the wavelet domain. For each set of PA data, a set of discriminative features are computed as a feature vector. When the tumor properties change as the condition varies (e.g., as a function of time following treatment), the corresponding PA signals will also diverge as will the statistical features of the signals. Therefore, the similarities and dissimilarities between the statistical feature can be used to identify and classify the changes in tissue as the tumor responds to the treatment. To visualize the similarities and dissimilarities, a computed similarity metric is mapped to a multidimensional space, such as a two- or three-dimensional space. In this produced map, each point represents the tumor in a particular condition. Points that are farther apart in the multidimensional space indicate more changes in tumor tissue due to changing condition and vise versa.

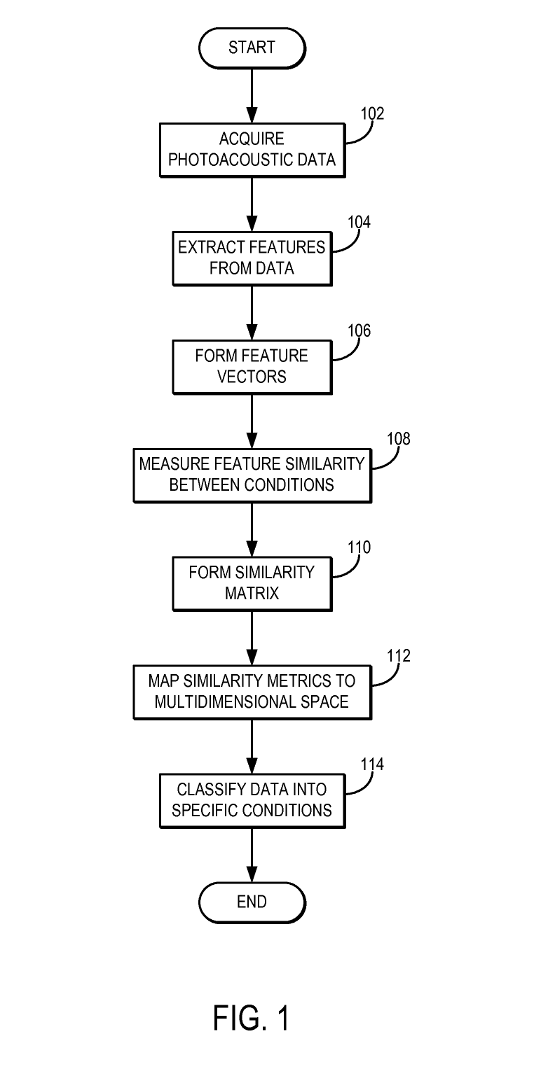

Referring now to FIG. 1, a flowchart setting forth the steps of an example of a method for classifying tissue response to a cancer treatment by analyzing photoacoustic signals is illustrated. The method begins with the acquisition of photoacoustic data from the tissue-of-interest, as indicated at step 102. The tissue-of-interest may form a part of a living subject or be an in vitro tissue sample. Photoacoustic data is acquired using an appropriate photoacoustic measurement system, such as a photoacoustic spectroscopy or photoacoustic imaging system, to scan the tissue at different time intervals. The photoacoustic data are acquired for a number, p, of different conditions, such as pre-treatment and post-treatment conditions. There may be multiple different conditions sampled during or after treatment. The photoacoustic data acquired for each condition includes a number, q, of frames of data that are obtained from different positions in the tissue. For instance, the data frames correspond to different slices through the tissue. Each data frame may include N photoacoustic signals corresponding to N different channels of an ultrasound transducer array that forms a part of the photoacoustic imaging system.

A set of discriminative features is extracted from each data frame, as indicated at step 104. By way of example, the features may be extracted in the wavelet domain. Extracting features in the wavelet domain combines information of both time and frequency. Statistical features such as energy distribution, homogeneity, and entropy are considered to reveal the relevant information. As indicated at step 106, the features extracted from each data frame are combined to form a feature vector for that data frame.

It is contemplated that the properties of the tissue change as a function of the different conditions under which the photoacoustic signals are obtained, such as pre-treatment and post-treatment. Thus, the photoacoustic signals acquired for the different conditions differ, as do the features extracted from the data frames obtained for the different conditions. To exploit these differences, the similarity between feature vectors is computed, as indicated at step 108. By way of example, the similarity between feature vectors can be computed by calculating the distance, such as the Euclidean distance, between pairs of feature vectors obtained for the same data frame under different conditions. In this instance, feature vectors that are farther apart indicate more change in the tissue due to the treatment. As a result of computing the similarity between feature vectors, a similarity matrix is formed, as indicated at step 110.

To visualize the similarity between feature vectors, the computed similarity metrics are mapped into a multidimensional space, as indicated at step 112. By way of example, a multidimensional scaling (“MDS”) analysis may be used to map the similarity matrix to a two-dimensional or three-dimensional space. The MDS analysis provides a visual representation of the pattern of similarities or dissimilarities among the data frames. Using this approach, each data frame is represented by a single point in the selected multidimensional space. The points are arranged in this space so that the distances between pairs of points have the strongest possible relation to the similarities among the pairs of the data frames. That is, two similar frames are represented by two points that are close together, and two dissimilar frames are represented by two points that are farther apart. By applying MDS to the similarity matrix, p×q points in two-dimensional space are obtained, where each point corresponds to one data frame.

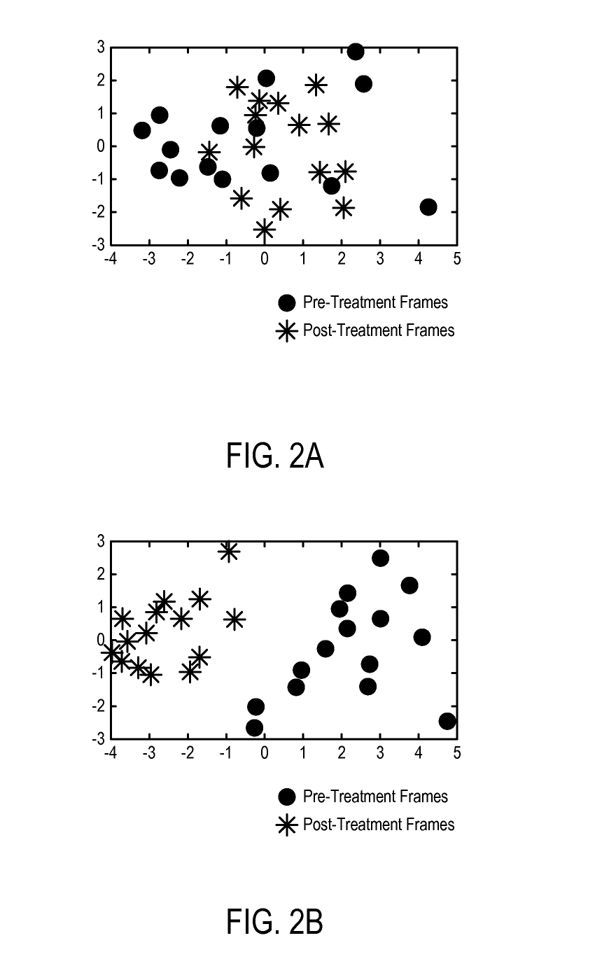

The data frames are classified into different groups according to the condition to which the data frames belong, as indicated at step 114. One way to classify the data frames is to use a clustering algorithm, such as a fuzzy c-means algorithm, on the points in the similarity treatment response map produced in step 112 in order to classify the points into different condition groups based on their similarity, such as pre-treatment and post-treatment groups. By applying a clustering algorithm, the points with more similarity are placed in the same cluster, and points that are significantly dissimilar from other groups of points are assigned to different a cluster. In some instances, p, clusters corresponding to p conditions may be obtained, but in other instances less clusters may be obtained. For example, when there is little dissimilarity between two different conditions, the points in each condition may not be assigned to two different clusters, but may instead be assigned to the same, single cluster. An example of this situation is illustrated in FIG. 2A, discussed below.

Examples of plots that depict the visualization of patterns in the proximity of data frames for given conditions are illustrated in FIGS. 2A and 2B. In these specific examples, the plots depict the results of applying the method of the present invention to a set of data obtained from tumor-bearing mice. FIG. 2A illustrates a control example, in which no treatment is applied to the subject, and FIG. 2B illustrates an example in which the subject is provided a cancer treatment, such as a radiation therapy treatment, an ultrasound treatment, a chemotherapy treatment, or other treatment type. In these examples, two data acquisition times were considered: before and after twenty-four hours. For each data acquisition time, fifteen frames of photoacoustic data were acquired, with each frame corresponding to a different slice location through a selected tumor. The frames obtained through scanning the tumor were considered as the samples of the particular condition. For each subject, the middle image frames, which contain tumor planes with the largest physical dimensions, were selected for processing. The algorithm described above was then applied on the beam-formed RF data of each frame within a region-of-interest that enclosed only tumor tissue.

FIGS. 2A and 2B depict the distances between the frames acquired at the two data acquisition times for a single subject (control in FIG. 2A and treated in FIG. 2B). As illustrated in FIG. 2A, there is a great amount of similarity between points acquired in the “pre-treatment” condition and points acquired in the “post-treatment” condition because no treatment was provided to the subject. On the contrary, as illustrated in FIG. 2B, when treatment is provided to the subject the data frames acquired during the pre-treatment and post-treatment conditions become significantly dissimilar. This dissimilarity is an indication that the applied treatment was effective, and the distance between the two conditions is representative of the degree of efficacy for the treatment. In this latter example, the frames are classified into two distinct clusters, or groups: one corresponding to a pre-treatment condition and one corresponding to a post-treatment condition. When more than two conditions are used, the relative distance between clusters of frames indicates the progress of treatment as the condition changes.

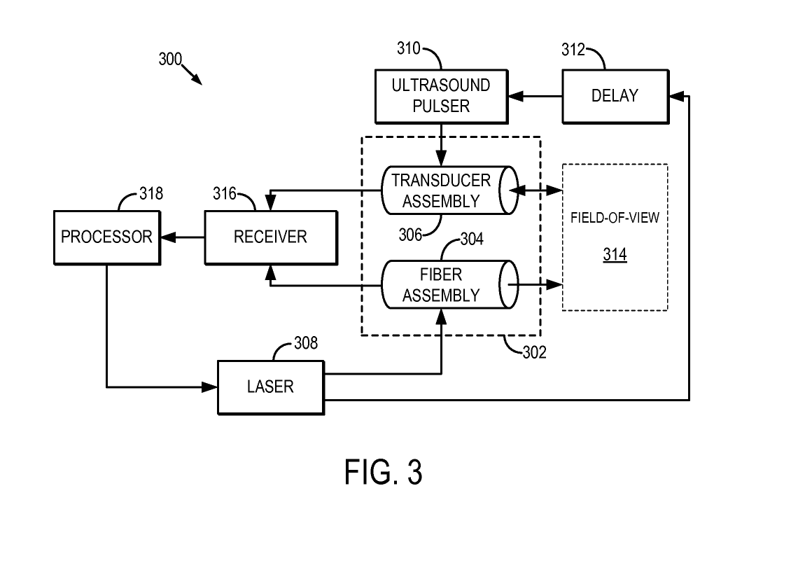

Referring now to FIG. 3, a block diagram of an example photoacoustic imaging system 300 that incorporates an photoacoustic imaging device 302 is illustrated. The photoacoustic imaging device 302 generally includes a fiber assembly 304 and a transducer assembly 306, which may be coupled together. For instance, the fiber assembly 304 and transducer assembly 306 may be coupled via a common outer sheath that holds the fiber assembly 304 and transducer assembly 306 in spaced arrangement.

The fiber assembly 304 includes at least one optical fiber. A light source 308 is optically coupled to the fiber assembly 304 and delivers light to the distal end of the fiber assembly 304 to irradiate the object being imaged. In some embodiments, the light source 308 is a laser source, which may be a continuous wave laser source or a pulse wave laser source. In some embodiments, the light source 308 may include multiple laser systems or diodes, thereby providing different optical wavelengths, that are fed into one or more optical fibers. The selection of an appropriate excitation wavelength for the light source 308 is based on the absorption characteristics of the imaging target. Because the average optical penetration depth for intravascular tissue is on the order of several to tens of millimeters, the 400-2100 nm wavelength spectral range is suitable for intravascular photoacoustic applications. Thus, in some embodiments, the light source 308 may be an Nd:YAG (neodymium-doped yttrium aluminum garnet) laser that operates at 1064 nm wavelength in a continuous mode.

The transducer assembly 306 generally includes an photoacoustic transducer for receiving photoacoustic signals generated by an illumination field, such as an illumination field generated by pulsed or continuous wave laser light. In some configurations, the photoacoustic transducer can also be operated to generate ultrasound energy and to receive pulse-echo ultrasound emissions. In this configuration, the photoacoustic transducer can be operated in a receive-only mode for photoacoustic imaging and, when the illumination field is not being generated, the photoacoustic transducer can also be operated in an ultrasound imaging mode to obtain ultrasound images. In some configurations, the photoacoustic transducer may include multiple transducer elements, some of which may be dedicated solely for receiving photoacoustic signals while others may be dedicated solely to generating and receiving pulse-echo ultrasound signals.

In some other configurations, the transducer assembly 306 may include at least two transducers: a dedicated photoacoustic transducer and a dedicated ultrasound transducer for generating and receiving pulse-echo ultrasound signals. In this dual-transducer configuration, both photoacoustic and ultrasound images can be obtained. With the dual-transducer configuration, photoacoustic and ultrasound images can be obtained simultaneously and, even when not obtained simultaneously, are innately co-registered given the spatial relationship between the photoacoustic transducer and the ultrasound transducer.

Irradiation with the light source 308 is performed at a given point for a finite amount of time with an optical excitation waveform. In frequency-domain photoacoustic applications, in which a continuous wave laser is used, the optical excitation waveform may be amplitude modulated with frequency sweeping, such as a chirp or pulse train. The irradiation produced by this type of optical excitation results in a frequency-domain photoacoustic modulated signal being produced in the region illuminated by the photoacoustic imaging device 302. The chirp can include a multitude of different excitation waveforms including linear, non-linear, and Gaussian tampered frequency swept chirps.

When used to obtain ultrasound images, operation of the transducer assembly 306 may be controlled by an ultrasound pulser 310, which provides ultrasound excitation waveforms to the transducer assembly 306. In single-transducer configurations in which the photoacoustic transducer is used to both receive photoacoustic signals and to generate and receive pulse-echo ultrasound signals, a delay 312 between the light source 308 and the ultrasound pulser 310 provides a trigger signal that directs the ultrasound pulser 310 to operate the photoacoustic transducer at a delay with respect to the irradiation of the field-of-view 314. The timing provided by the delay 312 enables the detection of photoacoustic signals by the photoacoustic transducer in the transducer assembly 306 when the field-of-view 314 is being illuminated, but also the generation and detection of pulse-echo ultrasound signals when the field-of-view 314 is not being illuminated.

Signals received by the transducer assembly 306 are communicated to a receiver 316, which generally includes a pre-amplifier, but may also include one or more filters, such as bandpass filters for signal conditioning. The received signals are then communicated to a processor 318 for analysis.

Thus, the generated photoacoustic signals are detected by the photoacoustic transducer in the transducer assembly 306, communicated to the receiver 316, and then communicated to the processor 318 for processing and/or image generation. As one example, the photoacoustic signals can be processed in accordance with the methods described above to monitor the physical changes in a tissue following the administration of a treatment, such as a radiation treatment or a chemotherapy treatment. Similarly, pulse-echo ultrasound signals received by either the photoacoustic transducer or a dedicated ultrasound transducer in the transducer assembly 306 can also be communicated to the receiver 316 and then communicated to the processor 318 for processing and/or image generation.

The present invention has been described in terms of one or more preferred embodiments, and it should be appreciated that many equivalents, alternatives, variations, and modifications, aside from those expressly stated, are possible and within the scope of the invention.

1. A method for monitoring physical changes in a tissue in response to a treatment using a photoacoustic measurement system, the steps of the method comprising:
a) acquiring photoacoustic data from at least one location within a tissue in a first condition using a photoacoustic measurement system;
b) acquiring photoacoustic data from the at least one location within the tissue in a second condition that is different than the first condition using the photoacoustic measurement system;
c) computing a similarity metric between the photoacoustic signals acquired in step a) and the photoacoustic signals acquired in step b);
d) producing a treatment response map by mapping the photoacoustic data acquired in steps a) and b) into a multidimensional space using the similarity metric computed in step c);
e) classifying the first condition and second condition as corresponding to a particular treatment response by comparing locations of the mapped photoacoustic data in the treatment response map.

2. The method as recited in claim 1 in which the first condition is a pre-treatment condition corresponding to a time before treatment is administered to the tissue and the second condition is a post-treatment condition corresponding to a time after treatment is administered to the tissue.

3. The method as recited in claim 1 in which step c) includes extracting statistical features from the photoacoustic data acquired in steps a) and b) and calculating, and in which the similarity metric is computed between the statistical features extracted from the photoacoustic data acquired in step a) and the statistical features extracted from the photoacoustic data acquired in step b).

4. The method as recited in claim 3 in which the statistical features include at least one of energy distribution, homogeneity, and entropy.

5. The method as recited in claim 3 in which the statistical features are extracted in a wavelet domain.

6. The method as recited in claim 3 in which the statistical features extracted from the photoacoustic data acquired in step a) are combined in a first feature vector and the statistical features extracted from the photoacoustic data acquired in step b) are combined in a second feature vector, and in which the similarity metric is computed between the first and second feature vector.

7. The method as recited in claim 6 in which the similarity metric computed in step c) is a Euclidean distance between the first and second feature vectors.

8. The method as recited in claim 1 in which step c) includes forming a similarity matrix from the computed similarity metrics, the similarity matrix having entries that correspond to a similarity between the first and second condition at different locations in the tissue.

9. The method as recited in claim 1 in which each point in the treatment response map produced in step d) corresponds to a different location in the tissue in at least one of the first and second conditions.

10. The method as recited in claim 9 in which step d) includes arranging each point in the tissue response map such that similar data are represented by points that are close together and dissimilar frames are represented by points that are farther apart.

11. The method as recited in claim 1 in which step e) includes applying a clustering algorithm to the treatment response map produced in step d).

12. The method as recited in claim 11 in which the clustering algorithm applied in step e) includes a fuzzy c-means algorithm.
